SEQUENCE LISTING (2 sequences)

SEQ ID NO: 1 - protein, 61 aa, synthetic construct
ADAQQNNFNKDQASAFYEILNMPNLNEAQRNGFIQSLKDDPSQSTNVLGEAKKLNESQAPK

SEQ ID NO: 2 - protein, 62 aa, synthetic construct
EVTIKVNLIFADGKIQTAEFKGTFEEATAEAYRYADLLAKVNGEYTADLEDGGNHMNIKFAG

(19) United States
(12) Patent Application Publication
(10) Pub. No.: US 2026/0043807 A1
(43) Pub. Date: Feb. 12, 2026

(54) PHOTOREACTIVE ANTIBODY-BINDING DOMAIN FOR SITE-SPECIFIC MODIFICATION OF THE ANTIGEN BINDING FRAGMENT (Fab) OF IgG AND IgM

(71) Applicants: THE TRUSTEES OF THE UNIVERSITY OF PENNSYLVANIA, Philadelphia, PA (US); ALPHATHERA, Philadelphia, PA (US)

(72) Inventors: Andrew TSOURKAS, Bryn Mawr, NY (US); Feifan YU, West Windsor, NJ (US)

(73) Assignees: THE TRUSTEES OF THE UNIVERSITY OF PENNSYLVANIA, Philadelphia, PA (US); ALPHATHERA, Philadelphia, PA (US)

(21) Appl. No.: 19/099,699

(22) PCT Filed: Aug. 8, 2023
(86) PCT No.: PCT/US2023/071882

§ 371 Date: Jan. 29, 2025

Related U.S. Application Data
(60) Provisional application No. 63/396,411, filed on Aug. 9, 2022.

(51) Int. Cl. G01N 33/574; C07K 16/28; G01N 33/532; G01N 33/543

(52) U.S. Cl. CPC G01N 33/57492; C07K 16/2863; G01N 33/532; G01N 33/54346; C07K 2317/55

(57) ABSTRACT

Provided is an adapter comprising an antibody binding domain (AbBD) that specifically binds and crosslinks to an immunoglobulin Fab region, where the AbBD is a variant of a Protein A domain D and has an amino acid replacement/modification at A28, G32, Q35, S36, N46, G49, E50, K53 or L54 of SEQ ID NO: 1 or combinations thereof, and where the AbBD is photoreactive. Also provided is an adapter comprising an AbBD that specifically binds and crosslinks to an IgG Fab region and/or to an immunoglobulin M (IgM) Fab region, where the AbBD is a variant of a Protein A domain D and has an amino acid replacement/modification at A28, E50, or L54 of SEQ ID NO: 1 or combinations thereof, where the AbBD is photoreactive. Provided is an adapter comprising an AbBD that specifically binds and crosslinks to an IgG Fab region, where the AbBD is a variant of a Protein L C* domain and has an amino acid replacement/modification at G22 or T46 of SEQ ID NO: 2 or a combination thereof, wherein the AbBD is photo-reactive. Methods for using the AbBD for imaging, diagnosing and/or treating a disease are provided.

### GOVERNMENT INTEREST STATEMENT

This invention was made with government support under EB023750 awarded by the National Institutes of Health. The United States government has certain rights in the invention.

### FIELD OF THE INVENTION

The invention relates to antibody conjugates, e.g., antibody-binding domains, drug and nanoparticle compositions and methods of generating the same. This invention further relates to methods of using the antibody conjugates, e.g., antibody-binding domains, drug and nanoparticle compositions in methods for imaging, diagnosing and/or treating a disease.

### BACKGROUND OF THE INVENTION

Photoreactive antibody-binding domains (pAbBDs) that bind to the Fc region of immunoglobulin G (IgG) and that form a covalent link with native IgG upon exposure to non-damaging long-wavelength UV light previously were created. The pAbBDs are composed of small protein adapters that are based on the structures of a single domain of Protein A or Protein G (or variants thereof). The pAbBDs possess a photocrosslinker within their Fc-binding region to enable crosslinking.

There still exists a need for site-selective conjugation of antibody-binding domains, antigen-binding fragments (Fabs), and variants thereof, e.g., which do not interfere with the photo-crosslinking of photoreactive antibody binding domains to the Fc domain, improved compositions comprising the site-specifically conjugated Fabs, and methods for imaging, diagnosing and/or treating a disease with the site-specifically conjugated immunoglobulin Fab domains.

### SUMMARY OF THE INVENTION

In one aspect, provided herein is an adapter comprising an antibody binding domain (AbBD) that specifically binds and crosslinks to an immunoglobulin Fab region, wherein the AbBD is a variant of a Protein A domain D comprising an amino acid sequence with at least 80% identity to the sequence set forth in SEQ ID NO: 1 and having an amino acid replacement and/or an amino acid modification at A28, G32, Q35, S36, N46, G49, E50, K53 or L54 of SEQ ID NO: 1 or combinations thereof that is photo-reactive.

In another aspect, provided herein is an adapter comprising an antibody binding domain (AbBD) that specifically binds and crosslinks to an immunoglobulin G (IgG) Fab region and to immunoglobulin M (IgM) Fab region, wherein the AbBD is a variant of a Protein A domain D comprising an amino acid sequence with at least 90% identity to the sequence set forth in SEQ ID NO: 1 and having an amino acid replacement and/or an amino acid modification at A28, E50, or L54 of SEQ ID NO: 1 or combinations thereof that is photo-reactive.

In yet another aspect, provided herein is an adapter comprising an antibody binding domain (AbBD) that specifically binds and crosslinks to an immunoglobulin Fab region, wherein the AbBD is a variant of a Protein L C* domain comprising an amino acid sequence with at least 80% identity to the sequence set forth in SEQ ID NO: 2 and having an amino acid replacement and/or an amino acid modification at G22 or T46 of SEQ ID NO: 2 or a combination thereof that is photo-reactive.

In one aspect, provided herein is a nucleic acid encoding the adapter according to claim 1, wherein the antibody binding domain (AbBD) comprises an amino acid sequence having at least 90% identity to the sequence set forth in SEQ ID NO: 1 and an amino acid replacement and/or an amino acid modification at A28, G32, Q35, S36, N46, G49, E50, K53 or L54 of SEQ ID NO: 1 or combinations thereof.

In another aspect, provided herein is a nucleic acid encoding the adapter according to claim 9, wherein the antibody binding domain (AbBD) comprises an amino acid sequence having at least 90% identity to the sequence set forth in SEQ ID NO: 1 and having an amino acid replacement and/or an amino acid modification at A28, E50, or L54 of SEQ ID NO: 1 or combinations thereof.

In still another aspect, provided herein is a nucleic acid encoding the adapter according to claim 15, wherein the antibody binding domain (AbBD) comprises an amino acid sequence with at least 90% identity to the sequence set forth in SEQ ID NO: 2 and having an amino acid replacement and/or an amino acid modification at G22 and T46 of SEQ ID NO: 2 or a combination thereof.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein.

FIG. 1 illustrates a photoreactive antibody-binding domains (pAbBD) that is able to site-specifically photo-crosslink to the IgG Fab domain, upon exposure to UV light.

FIGS. 2A-2B: FIG. 2A shows the amino acid sequence of the D domain of Staphylococcal Protein A. FIG. 2B shows an SDS-PAGE of Cetuximab (human IgG1) after photo-crosslinking with various photoreactive mutants the D domain of Protein A, under 365 nm UV illumination for 2 hours. The BPA positions tested are A28, Q29, G32, F33, Q35, S36, N46, G49, E50, K53 and L54. Appreciable photo-crosslinking was observed with A28, G32, Q35, S36, N46, G49, E50, K53 and L54.

FIG. 3 shows Protein A D domain and IgG conjugation by protein A D domain. Left side of gel (Lanes labeled 1-6): SDS-PAGE of human IgG1 (Cetuximab) alone or following photo-crosslinking with pAD #7 (N46BPA), Protein G HTB1 A24BPA, or pAD #7 (N46BPA) and Protein G HTB1 A24BPA. Both domains can bind and photo-crosslink to human IgG1 simultaneously, because pAD #7 (N46BPA) binds and photo-crosslinks the Fab fragment while Protein G HTB1 A24BPA photo-crosslinks the Fc domain. Right side of gels (Lanes labeled 1-11): SDS-PAGE of IgM alone (far right lane) or after photo-crosslinking with Protein A-D domain with BPA position introduced at various positions. The BPA positions tested are A28, Q29, G32, F33, Q35, S36, N46, G49, E50, K53 and L54. Protein A-D domain variants A28BPA, L54BPA, and E50BPA exhibit photo-crosslinking to IgM.

FIG. 4 shows protein A D domain E50BPA crosslinks to Cetuximab and human IgM: an SDS-PAGE of Cetuximab (human IgG1) and human IgM alone and after photo-crosslinking with protein A D domain E50BPA. protein A D domain E50BPA labeled ˜50% of the heavy chains of IgG and IgM.

FIGS. 5A-5B: FIG. 5A shows the amino acid sequence of protein L, C* domain. FIG. 5B shows an SDS-PAGE of Cetuximab (human IgG1) after photo-crosslinking with various photoreactive mutants of Protein L C* domain, under 365 nm UV illumination for 2 hrs. The BPA positions tested are Q16, T17, A18, G22, T23, E26, R33, Y34, A47, T46 and D48. FIG. 5B also shows that T46BPA can conjugate to human IgG1 with high efficiency. Photo-crosslinking also was observed with G22BPA.

FIG. 6 shows photo-crosslinking of IgGs from various host species (human, rabbit, goat) with a photoreactive pL C* domain with BPA inserted at site T46 (T46BPA). SDS-PAGE of IgG from different host species (human, rabbit, goat) alone and after photo-crosslinking with protein L T46*BPA. Photo-crosslinking was performed under 365 nm illumination for 2 hrs. The variant T46BPA of pL can label human IgG1 and Rabbit IgG with 80-100% conjugation efficiency and Goat IgG with ˜30-50% conjugation efficiency.

FIG. 7 shows photo-crosslinking of IgGs from various host species and subclasses with a photoreactive pL C* domain with BPA inserted at site T46 (T46BPA). SDS-PAGE of IgG from different host species (Cetuximab/human IgG1, Herceptin/human IgG1, mouse IgG2a, mouse IgG2b, Rabbit IgG, Goat IgG and Rat IgG) alone and after photo-crosslinking with protein L T46*BPA. Photo-crosslinking was performed under 365 nm illumination for 2 hours. The variant T46BPA can label antibodies from a wide range of hosts and subclasses.

FIG. 8 shows photo-crosslinking of IgGs from various host species and subclasses (human IgG1, mouse IgG2a and 2b, rabbit, goat, and rat) with a photoreactive pL C* domain with BPA inserted at site G22 (G22BPA). SDS-PAGE of IgG from different host species (Cetuximab/human IgG1, mouse IgG2a, mouse IgG2b, Rabbit IgG, Goat IgG and Rat IgG) alone and after photo-crosslinking with protein L G22*BPA. Photo-crosslinking was performed under 365 nm illumination for 2 hours. The variant G22BPA can label human IgG1 and Rabbit IgG with >50% conjugation efficiency.

### DETAILED DESCRIPTION OF THE INVENTION

In the following detailed description, numerous specific details are set forth in order to provide a thorough understanding of the invention. However, it will be understood by those skilled in the art that this disclosure is not limited to the specific products, methods, conditions or parameters described and/or shown herein, and that the terminology used herein is for the purpose of describing particular embodiments by way of example only and is not intended to be limiting of the claimed disclosure. In other instances, well-known methods, procedures, and components have not been described in detail so as not to obscure the present invention.

The present invention addresses the need for site-selective conjugation of antibody-binding domains, antigen-binding fragments (Fabs), and variants thereof, improved compositions comprising the site-specifically conjugated Fabs, and methods for imaging, diagnosing and/or treating a disease with the site-specifically conjugated immunoglobulin Fab domains, by providing site-specifically modified protein variants having photoreactive amino acid(s) incorporated at specific sites, which sites allow for photo-crosslinking to immunoglobulins from various hosts.

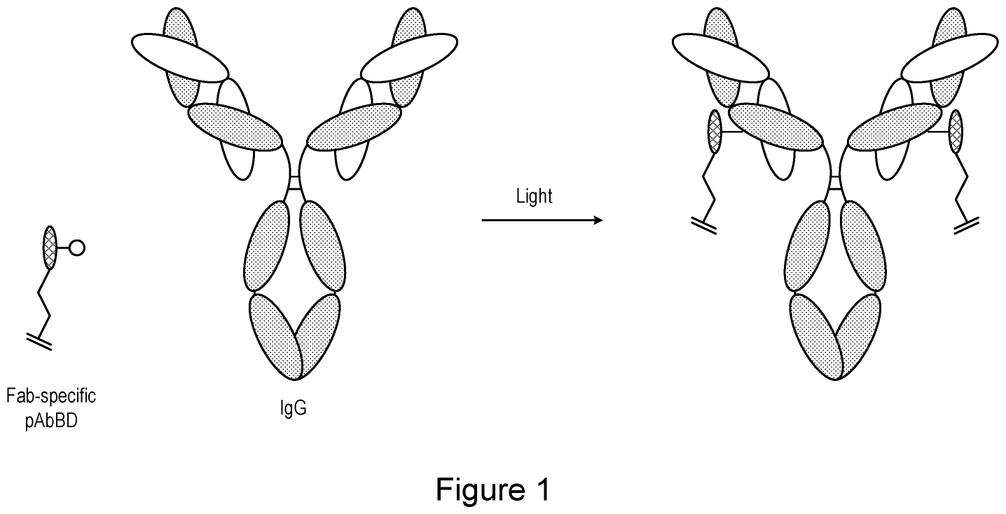

To expand upon the versatility of photoreactive antibody-binding domains (pAbBDs), the inventors prepared multiple variants of the D domain of protein A (also referred to herein as Protein A D domain) and identified variants that are able to covalently label the Fab domain of IgG, upon exposure to UV light (having BPA incorporation sites A28, G32, Q35, S36, N46, G49, E50, K53 and L54 of SEQ ID NO: 1) (FIG. 1). These sites allow photo-crosslinking to IgG from various hosts. They specifically label the Fab domain of IgG and do not interfere with the photo-crosslinking of photoreactive antibody binding domains to the Fc domain. Three of these variants, A28, E50, and L54, are also able to covalently label IgM with high efficiency. These Fab-specific pAbBDs can covalently label IgG simultaneously with Fc-specific pAbBDs and the A28, E50, and L54 variants are the first pAbBDs that are able to covalently label IgM. Like other pAbBDs, the Fab-specific pAbBD can be produced with a bioorthogonal chemical handle (e.g., biotin, azides, alkynes, thiols, etc.), fluorescent labels, drugs, polymers, or oligonucleotides at the c-terminus or fused to enzymes/proteins/peptides. This allows for site-specific and covalent immobilization of the pAbBD (and bound antibody or antibody fragment) onto a wide range of surfaces or the facile functionalization of IgG/IgM (or fragment) with other desirable biological or chemical moieties. The pAbBDs can be produced in an entirely recombinant manner using only minor variations of standard protein expression and purification protocols or made synthetically, via standard peptide synthesis methods. Both approaches are cost-effective and scalable.

The inventors also prepared variants of the C* domain of protein L (also referred to herein as Protein L C* domain) that covalently label the Fab domain of IgG, upon exposure to UV light (having BPA incorporation sites G22 and T46). These sites allow photo-crosslinking to IgG from various hosts.

In one aspect, provided herein is an adapter comprising an antibody binding domain (AbBD) that specifically binds and crosslinks to an immunoglobulin Fab region, wherein the AbBD is a variant of a Protein A domain D comprising an amino acid sequence with at least 80% identity to the sequence set forth in SEQ ID NO: 1 and having an amino acid replacement and/or an amino acid modification at A28, G32, Q35, S36, N46, G49, E50, K53 or L54 of SEQ ID NO: 1 or combinations thereof that is photo-reactive. In an embodiment of the adapter, the photo-reactive amino acid replacement is benzoylphenylalanine (BPA). In some embodiments, a cysteine is engineered into the AbBD and is modified with a photo-reactive moiety. In some embodiments, the Fab-specific pAbBD is conjugated to a protein, a peptide, a targeting agent of interest, an oligonucleotide, a DNA, an RNA, a detectable label, an imaging agent, a drug, a nanoparticle, a toxin, a hapten, a chelate, a polymer, a chemical handle, or a combination thereof. In an embodiment, the chemical handle is a click chemistry group selected from the group consisting of an azide, an alkyne, a constrained alkyne, dibenzocyclooctyne, a tetrazine, biotin, a thiol, and a transcyclooctene. In some embodiments, the immunoglobulin is an IgG molecule. In a particular embodiment, the Protein A domain D comprises an amino acid sequence with at least 80% identity to the sequence set forth in SEQ ID NO: 1 and has an amino acid replacement and/or an amino acid modification at A28, E50, or L54 of SEQ ID NO: 1 or combinations thereof that is photo-reactive. In an embodiment, the immunoglobulin is an IgG molecule and/or an IgM. In a particular embodiment, the pAbBD is pADE50BPA, which photo-crosslinks IgM.

In another aspect, provided herein is an adapter comprising an antibody binding domain (AbBD) that specifically binds and crosslinks to an immunoglobulin G (IgG) Fab region and to immunoglobulin M (IgM) Fab region, wherein the AbBD is a variant of a Protein A domain D comprising an amino acid sequence with at least 90% identity to the sequence set forth in SEQ ID NO: 1 and having an amino acid replacement and/or an amino acid modification at A28, E50, or L54 of SEQ ID NO: 1 or a combination thereof that is photo-reactive. In some embodiments of the adapter, the photo-reactive amino acid replacement is benzoylphenylalanine (BPA). In certain embodiments, a cysteine is engineered into the AbBD and is modified with a photo-reactive moiety. In various embodiments, the Fab-specific pAbBD is conjugated to a protein, a peptide, a targeting agent of interest, an oligonucleotide, a DNA, an RNA, a detectable label, an imaging agent, a drug, a nanoparticle, a toxin, a hapten, a chelate, a polymer, a chemical handle, or a combination thereof. In an embodiment, the chemical handle is a click chemistry group selected from the group consisting of an azide, an alkyne, a constrained alkyne, dibenzocyclooctyne, a tetrazine, biotin, a thiol, and a transcyclooctene. In some embodiments, the immunoglobulin is an IgG molecule and/or an IgM. In certain embodiments, the pAbBD is pADE50BPA, which photo-crosslinks IgM.

In yet another aspect, provided herein is an adapter comprising an antibody binding domain (AbBD) that specifically binds and crosslinks to an immunoglobulin Fab region, wherein the AbBD is a variant of a Protein L C* domain comprising an amino acid sequence with at least 80% identity to the sequence set forth in SEQ ID NO: 2 or a combination thereof and having an amino acid replacement and/or an amino acid modification at G22 or T46 of SEQ ID NO: 2 that is photo-reactive. In some embodiments of the adapter, the photo-reactive amino acid replacement is benzoylphenylalanine (BPA). In an embodiment, a cysteine is engineered into the AbBD and is modified with a photo-reactive moiety. In certain embodiments the Fab-specific pAbBD is conjugated to a protein, a peptide, a targeting agent of interest, an oligonucleotide, a DNA, an RNA, a detectable label, an imaging agent, a drug, a nanoparticle, a toxin, a hapten, a chelate, a polymer, a chemical handle, or a combination thereof. In some embodiments the chemical handle is a click chemistry group selected from the group consisting of an azide, an alkyne, a constrained alkyne, dibenzocyclooctyne, a tetrazine, biotin, a thiol, and a transcyclooctene.

In one aspect, provided herein is a nucleic acid encoding an adapter comprising an antibody binding domain (AbBD) that specifically binds and crosslinks, wherein the antibody binding domain (AbBD) comprises an amino acid sequence having at least 80% identity to the sequence set forth in SEQ ID NO: 1 and an amino acid replacement and/or an amino acid modification at A28, G32, Q35, S36, N46, G49, E50, K53 or L54 of SEQ ID NO: 1 or combinations thereof. In some embodiments, provided is a nucleic acid encoding an adapter comprising an antibody binding domain (AbBD) that specifically binds and crosslinks, wherein the antibody binding domain (AbBD) comprises an amino acid sequence having at least 90% identity to the sequence set forth in SEQ ID NO: 1 and an amino acid replacement and/or an amino acid modification at A28, G32, Q35, S36, N46, G49, E50, K53 or L54 of SEQ ID NO: 1 or combinations thereof. In various embodiments, provided is a nucleic acid encoding an adapter comprising an antibody binding domain (AbBD) that specifically binds and crosslinks, wherein the antibody binding domain (AbBD) comprises an amino acid sequence having the sequence set forth in SEQ ID NO: 1 and an amino acid replacement and/or an amino acid modification at A28, G32, Q35, S36, N46, G49, E50, K53 or L54 of SEQ ID NO: 1 or combinations thereof.

In certain embodiments, an expression vector comprises a nucleic acid encoding an amino acid sequence having at least 80% identity to the sequence set forth in SEQ ID NO: 1 and having an amino acid replacement and/or an amino acid modification at A28, G32, Q35, S36, N46, G49, E50, K53 or L54 of SEQ ID NO: 1 or combinations thereof. In an embodiment, an expression vector comprises a nucleic acid encoding an amino acid sequence having at least 90% identity to the sequence set forth in SEQ ID NO: 1 and having an amino acid replacement and/or an amino acid modification at A28, G32, Q35, S36, N46, G49, E50, K53 or L54 of SEQ ID NO: 1 or combinations thereof. In some embodiments, an expression vector comprises a nucleic acid encoding an amino acid sequence having the sequence set forth in SEQ ID NO: 1 and having an amino acid replacement and/or an amino acid modification at A28, G32, Q35, S36, N46, G49, E50, K53 or L54 of SEQ ID NO: 1 or combinations thereof.

In particular embodiments, a host cell comprises the expression vector comprising a nucleic acid encoding an amino acid sequence having at least 80% identity to the sequence set forth in SEQ ID NO: 1 and having an amino acid replacement and/or an amino acid modification at A28, G32, Q35, S36, N46, G49, E50, K53 or L54 of SEQ ID NO: 1 or combinations thereof. In some embodiments, a host cell comprises the expression vector comprising a nucleic acid encoding an amino acid sequence having at least 90% identity to the sequence set forth in SEQ ID NO: 1 and having an amino acid replacement and/or an amino acid modification at A28, G32, Q35, S36, N46, G49, E50, K53 or L54 of SEQ ID NO: 1 or combinations thereof. In additional embodiments, a host cell comprises the expression vector comprising a nucleic acid encoding an amino acid sequence having the sequence set forth in SEQ ID NO: 1 and having an amino acid replacement and/or an amino acid modification at A28, G32, Q35, S36, N46, G49, E50, K53 or L54 of SEQ ID NO: 1 or combinations thereof.

In another aspect, provided herein is a nucleic acid encoding an adapter comprising an antibody binding domain (AbBD) that specifically binds and crosslinks, wherein the antibody binding domain (AbBD) comprises an amino acid sequence having at least 80% identity to the sequence set forth in SEQ ID NO: 1 and having an amino acid replacement and/or an amino acid modification at A28, E50, or L54 of SEQ ID NO: 1 or combinations thereof. In some embodiments, provided is a nucleic acid encoding an adapter comprising an antibody binding domain (AbBD) that specifically binds and crosslinks, wherein the antibody binding domain (AbBD) comprises an amino acid sequence having at least 90% identity to the sequence set forth in SEQ ID NO: 1 and having an amino acid replacement and/or an amino acid modification at A28, F50, or 1.54 of SEQ ID NO: 1 or combinations thereof. In various embodiments, provided is a nucleic acid encoding an adapter comprising an antibody binding domain (AbBD) that specifically binds and crosslinks, wherein the antibody binding domain (AbBD) comprises an amino acid sequence having the sequence set forth in SEQ ID NO: 1 and having an amino acid replacement and/or an amino acid modification at A28. E50, or L54 of SEQ ID NO: 1 or combinations thereof.

In various embodiments, an expression vector comprises a nucleic acid encoding an amino acid sequence having at least 80% identity to the sequence set forth in SEQ ID NO: 1 and having an amino acid replacement and/or an amino acid modification at A28, E50, or L54 of SEQ ID NO: 1 or combinations thereof. In an embodiment, an expression vector comprises a nucleic acid encoding an amino acid sequence having at least 90% identity to the sequence set forth in SEQ ID NO: 1 and having an amino acid replacement and/or an amino acid modification at A28, E50, or L54 of SEQ ID NO: 1 or combinations thereof. In some embodiments, an expression vector comprises a nucleic acid encoding an amino acid sequence having the sequence set forth in SEQ ID NO: 1 and having an amino acid replacement and/or an amino acid modification at A28, E50, or L54 of SEQ ID NO: 1 or combinations thereof.

In some embodiments, a host cell comprises the expression vector comprising a nucleic acid encoding an amino acid sequence having at least 80% identity to the sequence set forth in SEQ ID NO: 1 and having an amino acid replacement and/or an amino acid modification at A28, E50, or L54 of SEQ ID NO: 1 or combinations thereof. In various embodiments, a host cell comprises the expression vector comprising a nucleic acid encoding an amino acid sequence having at least 90% identity to the sequence set forth in SEQ ID NO: 1 and having an amino acid replacement and/or an amino acid modification at A28, E50, or L54 of SEQ ID NO: 1 or combinations thereof. In additional embodiments, a host cell comprises the expression vector comprising a nucleic acid encoding an amino acid sequence having the sequence set forth in SEQ ID NO: 1 and having an amino acid replacement and/or an amino acid modification at A28, E50, or L54 of SEQ ID NO: 1 or combinations thereof.

In one aspect, provided herein is a nucleic acid encoding an adapter comprising an antibody binding domain (AbBD) that specifically binds and crosslinks, wherein the antibody binding domain (AbBD) comprises an amino acid sequence having at least 80% identity to the sequence set forth in SEQ ID NO: 2 and having an amino acid replacement and/or an amino acid modification at G22 or T46 of SEQ ID NO: 2 or a combination thereof. In some embodiments, provided is a nucleic acid encoding an adapter comprising an antibody binding domain (AbBD) that specifically binds and crosslinks, wherein the antibody binding domain (AbBD) comprises an amino acid sequence having at least 90% identity to the sequence set forth in SEQ ID NO: 2 and having an amino acid replacement and/or an amino acid modification at G22 or T46 of SEQ ID NO: 2 or a combination thereof. In various embodiments, provided is a nucleic acid encoding an adapter comprising an antibody binding domain (AbBD) that specifically binds and crosslinks, wherein the antibody binding domain (AbBD) comprises an amino acid sequence having the sequence set forth in SEQ ID NO: 2 and having an amino acid replacement and/or an amino acid modification at G22 or T46 of SEQ ID NO: 2 or a combination thereof.

In certain embodiments, an expression vector comprises a nucleic acid encoding an amino acid sequence having at least 80% identity to the sequence set forth in SEQ ID NO: 2 and having an amino acid replacement and/or an amino acid modification at G22 or T46 of SEQ ID NO: 2 or a combination thereof. In an embodiment, an expression vector comprises a nucleic acid encoding an amino acid sequence having at least 90% identity to the sequence set forth in SEQ ID NO: 2 and having an amino acid replacement and/or an amino acid modification at G22 or T46 of SEQ ID NO: 2 or a combination thereof. In another embodiment, an expression vector comprises a nucleic acid encoding an amino acid sequence having the sequence set forth in SEQ ID NO: 2 and having an amino acid replacement and/or an amino acid modification at G22 or T46 of SEQ ID NO: 2 or a combination thereof.

In an embodiment, a host cell comprises the expression vector comprising a nucleic acid encoding an amino acid sequence having at least 80% identity to the sequence set forth in SEQ ID NO: 2 and having an amino acid replacement and/or an amino acid modification at G22 or T46 of SEQ ID NO: 2 or a combination thereof. In various embodiments, a host cell comprises the expression vector comprising a nucleic acid encoding an amino acid sequence having at least 90% identity to the sequence set forth in SEQ ID NO: 2 and having an amino acid replacement and/or an amino acid modification at G22 or T46 of SEQ ID NO: 2 or a combination thereof. In some embodiments, a host cell comprises the expression vector comprising a nucleic acid encoding an amino acid sequence having the sequence set forth in SEQ ID NO: 2 and having an amino acid replacement and/or an amino acid modification at G22 or T46 of SEQ ID NO: 2 or a combination thereof.

In some embodiments, provided herein is a nanoparticle conjugate comprising the adapter comprising an antibody binding domain (AbBD) that specifically binds and crosslinks to an immunoglobulin Fab region, wherein the AbBD is a variant of a Protein A domain D comprising an amino acid sequence with at least 80% identity to the sequence set forth in SEQ ID NO: 1 and having an amino acid replacement and/or an amino acid modification at A28, G32, Q35, S36, N46, G49, E50, K53 or L54 of SEQ ID NO: 1 or combinations thereof that is photo-reactive. In an embodiment, a nanoparticle conjugate comprises an adapter comprising an antibody binding domain (AbBD) that specifically binds and crosslinks to an immunoglobulin Fab region, wherein the AbBD is a variant of a Protein A domain D comprising an amino acid sequence with at least 90% identity to the sequence set forth in SEQ ID NO: 1 and having an amino acid replacement and/or an amino acid modification at A28, G32, Q35, S36, N46, G49, E50, K53 or L54 of SEQ ID NO: 1 or combinations thereof that is photo-reactive. In some embodiments, a nanoparticle conjugate comprises an adapter comprising an antibody binding domain (AbBD) that specifically binds and crosslinks to an immunoglobulin Fab region, wherein the AbBD is a variant of a Protein A domain D comprising an amino acid sequence set forth in SEQ ID NO: 1 and having an amino acid replacement and/or an amino acid modification at A28, G32, Q35, S36, N46, G49, E50, K53 or L54 of SEQ ID NO: 1 or combinations thereof that is photo-reactive.

In various embodiments, provided herein is a nanoparticle conjugate comprising the adapter comprising an antibody binding domain (AbBD) that specifically binds and crosslinks to an immunoglobulin G (IgG) Fab region and to immunoglobulin M (IgM) Fab region, wherein the AbBD is a variant of a Protein A domain D comprising an amino acid sequence with at least 80% identity to the sequence set forth in SEQ ID NO: 1 and having an amino acid replacement and/or an amino acid modification at A28, E50, or L54 of SEQ ID NO: 1 or combinations thereof that is photo-reactive. In some embodiments, a nanoparticle conjugate comprises the adapter comprising an antibody binding domain (AbBD) that specifically binds and crosslinks to an immunoglobulin G (IgG) Fab region and to immunoglobulin M (IgM) Fab region, wherein the AbBD is a variant of a Protein A domain D comprising an amino acid sequence with at least 90% identity to the sequence set forth in SEQ ID NO: 1 and having an amino acid replacement and/or an amino acid modification at A28, E50, or L54 of SEQ ID NO: 1 or combinations thereof that is photo-reactive. In an embodiment, a nanoparticle conjugate comprises an adapter comprising an antibody binding domain (AbBD) that specifically binds and crosslinks to an immunoglobulin G (IgG) Fab region and to immunoglobulin M (IgM) Fab region, wherein the AbBD is a variant of a Protein A domain D comprising an amino acid sequence set forth in SEQ ID NO: 1 and having an amino acid replacement and/or an amino acid modification at A28, E50, or L54 of SEQ ID NO: 1 or combinations thereof that is photo-reactive. In a particular embodiment, the pAbBD is pADE50BPA, which photo-crosslinks IgM.

In some embodiments, provided herein is a nanoparticle conjugate comprising an adapter comprising an antibody binding domain (AbBD) that specifically binds and crosslinks to an immunoglobulin Fab region, wherein the AbBD is a variant of a Protein L C* domain comprising an amino acid sequence with at least 80% identity to the sequence set forth in SEQ ID NO: 2 and having an amino acid replacement and/or an amino acid modification at G22 or T46 of SEQ ID NO: 2 or a combination thereof that is photo-reactive. In an embodiment, a nanoparticle conjugate comprises an adapter comprising an antibody binding domain (AbBD) that specifically binds and crosslinks to an immunoglobulin Fab region, wherein the AbBD is a variant of a Protein L C* domain comprising an amino acid sequence with at least 90% identity to the sequence set forth in SEQ ID NO: 2 and having an amino acid replacement and/or an amino acid modification at G22 or T46 of SEQ ID NO: 2 or a combination thereof that is photo-reactive. In certain embodiments, the nanoparticle conjugate comprises an adapter comprising an antibody binding domain (AbBD) that specifically binds and crosslinks to an immunoglobulin Fab region, wherein the AbBD is a variant of a Protein L C* domain comprising an amino acid sequence set forth in SEQ ID NO: 2 and having an amino acid replacement and/or an amino acid modification at G22 or T46 of SEQ ID NO: 2 or a combination thereof that is photo-reactive.

In one aspect, provided herein is a method for imaging a biological sample from a subject, the method comprising contacting the biological sample with a nanoparticle conjugate comprising an adapter comprising an antibody binding domain (AbBD) that specifically binds and crosslinks to an immunoglobulin Fab region, wherein the AbBD is a variant of a Protein A domain D comprising an amino acid sequence with at least 80% identity to the sequence set forth in SEQ ID NO: 1 and having an amino acid replacement and/or an amino acid modification at A28, G32, Q35, S36, N46, G49, E50, K53 or L54 of SEQ ID NO: 1 or combinations thereof that is photo-reactive, wherein the contacting permits imaging of the biological sample. In an embodiment of the method for imaging a biological sample from a subject, the method comprising contacting the biological sample with a nanoparticle conjugate comprising an adapter comprising an antibody binding domain (AbBD) that specifically binds and crosslinks to an immunoglobulin Fab region, wherein the AbBD is a variant of a Protein A domain D comprising an amino acid sequence with at least 90% identity to the sequence set forth in SEQ ID NO: 1 and having an amino acid replacement and/or an amino acid modification at A28, G32, Q35, S36, N46, G49, E50, K53 or L54 of SEQ ID NO: 1 or combinations thereof that is photo-reactive, wherein the contacting permits imaging of the biological sample. In some embodiments of the method for imaging a biological sample from a subject, the method comprising contacting the biological sample with a nanoparticle conjugate comprising an adapter comprising an antibody binding domain (AbBD) that specifically binds and crosslinks to an immunoglobulin Fab region, wherein the AbBD is a variant of a Protein A domain D comprising an amino acid sequence set forth in SEQ ID NO: 1 and having an amino acid replacement and/or an amino acid modification at A28, G32, Q35, S36, N46, G49, E50, K53 or L54 of SEQ ID NO: 1 or combinations thereof that is photo-reactive, wherein the contacting permits imaging of the biological sample. In various embodiments of the herein provided methods for imaging a biological sample from a subject, the biological sample is associated with a disease. In some embodiments of said methods, imaging the biological sample permits diagnosing the disease. In certain embodiments of said methods, the biological sample is a tissue, DNA, sperm, sera, blood, cerebrospinal fluid, a tumor, or a biomarker. In particular embodiments, wherein when the imaging shows abnormal tissue or the tumor, said methods further comprise treating the subject with surgery, chemotherapy, radiation therapy, bone marrow transplant, immunotherapy, hormone therapy, targeted drug therapy, cryoablation, and/or radiofrequency ablation.

In another aspect, provided herein is a method for imaging a biological sample from a subject, the method comprising contacting the biological sample with a nanoparticle conjugate comprising an adapter comprising an antibody binding domain (AbBD) that specifically binds and crosslinks to an immunoglobulin G (IgG) Fab region and to immunoglobulin M (IgM) Fab region, wherein the AbBD is a variant of a Protein A domain D comprising an amino acid sequence with at least 80% identity to the sequence set forth in SEQ ID NO: 1 and having an amino acid replacement and/or an amino acid modification at A28, E50, or L54 of SEQ ID NO: 1 or combinations thereof that is photo-reactive, where the contacting permits imaging of the biological sample. In some embodiments of the method for imaging a biological sample from a subject, the method comprises contacting the biological sample with a nanoparticle conjugate comprising an adapter comprising an antibody binding domain (AbBD) that specifically binds and crosslinks to an immunoglobulin G (IgG) Fab region and to immunoglobulin M (IgM) Fab region, wherein the AbBD is a variant of a Protein A domain D comprising an amino acid sequence with at least 90% identity to the sequence set forth in SEQ ID NO: 1 and having an amino acid replacement and/or an amino acid modification at A28, E50, or L54 of SEQ ID NO: 1 or combinations thereof that is photo-reactive, wherein the contacting permits imaging of the biological sample. In certain embodiments of the method for imaging a biological sample from a subject, the method comprises contacting the biological sample with a nanoparticle conjugate comprising an adapter comprising an antibody binding domain (AbBD) that specifically binds and crosslinks to an immunoglobulin G (IgG) Fab region and to immunoglobulin M (IgM) Fab region, wherein the AbBD is a variant of a Protein A domain D comprising an amino acid sequence set forth in SEQ ID NO: 1 and having an amino acid replacement and/or an amino acid modification at A28, E50, or 1.54 of SEQ ID NO: 1 or combinations thereof, that is photo-reactive, wherein the contacting permits imaging of the biological sample. In various embodiments of the herein provided methods for imaging a biological sample from a subject, the biological sample is associated with a disease. In some embodiments of said methods, imaging the biological sample permits diagnosing the disease. In certain embodiments of said methods, the biological sample is a tissue, DNA, sperm, sera, blood, cerebrospinal fluid, a tumor, or a biomarker. In particular embodiments, wherein when the imaging shows abnormal tissue or the tumor, said methods further comprise treating the subject with surgery, chemotherapy, radiation therapy, bone marrow transplant, immunotherapy, hormone therapy, targeted drug therapy, cryoablation, and/or radiofrequency ablation.

In another aspect, provided herein is a method for imaging a biological sample from a subject, the method comprising contacting the biological sample with a nanoparticle conjugate comprising an adapter comprising an antibody binding domain (AbBD) that specifically binds and crosslinks to an immunoglobulin Fab region, wherein the AbBD is a variant of a Protein L C* domain comprising an amino acid sequence with at least 80% identity to the sequence set forth in SEQ ID NO: 2 and having an amino acid replacement and/or an amino acid modification at G22 or T46 of SEQ ID NO: 2 or a combinations thereof that is photo-reactive, where the contacting permits imaging of the biological sample. In certain embodiments of the method for imaging a biological sample from a subject, the method comprises contacting the biological sample with a nanoparticle conjugate comprising an adapter comprising an antibody binding domain (AbBD) that specifically binds and crosslinks to an immunoglobulin Fab region, wherein the AbBD is a variant of a Protein L C* domain comprising an amino acid sequence with at least 90% identity to the sequence set forth in SEQ ID NO: 2 and having an amino acid replacement and/or an amino acid modification at G22 or T46 of SEQ ID NO: 2 or a combination thereof that is photo-reactive, where the contacting permits imaging of the biological sample. In various embodiments of the method for imaging a biological sample from a subject, the method comprises contacting the biological sample with a nanoparticle conjugate comprising an adapter comprising an antibody binding domain (AbBD) that specifically binds and crosslinks to an immunoglobulin Fab region, wherein the AbBD is a variant of a Protein L C* domain comprising an amino acid sequence set forth in SEQ ID NO: 2 and having an amino acid replacement and/or an amino acid modification at G22 or T46 of SEQ ID NO: 2 or a combination thereof that is photo-reactive, wherein the contacting permits imaging of the biological sample. In particular embodiments of the herein provided methods for imaging a biological sample from a subject, the biological sample is associated with a disease. In various embodiments of said methods, imaging the biological sample permits diagnosing the disease. In some embodiments of said methods, the biological sample is a tissue, DNA, sperm, sera, blood, cerebrospinal fluid, a tumor, or a biomarker. In certain embodiments, wherein when the imaging shows abnormal tissue or the tumor, said methods further comprise treating the subject with surgery, chemotherapy, radiation therapy, bone marrow transplant, immunotherapy, hormone therapy, targeted drug therapy, cryoablation, and/or radiofrequency ablation.

Unless otherwise defined herein, scientific and technical terms used in connection with this disclosure shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In this disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

Whenever a numerical range is indicated herein, it is meant to include any cited numeral (fractional or integral) within the indicated range. The phrases “ranging/ranges between” a first indicated number and a second indicated number and “ranging/ranges from” a first indicated number “to” a second indicated number are used herein interchangeably and are meant to include the first and second indicated numbers and all the fractional and integral numerals therebetween.

When values are expressed as approximations, by use of the antecedent “about,” it is understood that the particular value forms another embodiment. All ranges are inclusive and combinable. In one embodiment, the term “about” refers to a deviance of between 0.1-5% from the indicated number or range of numbers.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, when referring to a measurable value such as an amount, a temporal duration, a concentration, and the like, may encompass variations of ±20% or ±10%, more specifically ±5%, even more particularly ±1%, and still more preferably +0.1% from the specified value, as such variations are appropriate to perform the disclosed methods. In another embodiment, the term “about” refers to a deviance of between 1-10% from the indicated number or range of numbers. In another embodiment, the term “about” refers to a deviance of up to 20% from the indicated number or range of numbers. In one embodiment, the term “about” refers to a deviance of ±10% from the indicated number or range of numbers. In another embodiment, the term “about” refers to a deviance of ±5% from the indicated number or range of numbers.

As used herein, the term “nucleic acid” refers to polynucleotides or to oligonucleotides such as deoxyribonucleic acid (DNA), and, where appropriate, ribonucleic acid (RNA) or mimetics thereof. This term should also be understood to include, as equivalents, analogs of either RNA or DNA made from nucleotide analogs, and, as applicable to the embodiment being described, single (sense or antisense) and double-stranded polynucleotides. This term includes oligonucleotides composed of naturally occurring nucleobases, sugars and covalent internucleoside (backbone) linkages as well as oligonucleotides having non-naturally-occurring portions, which function similarly. Such modified or substituted oligonucleotides may be used in place of native forms of oligonucleotides because of desirable properties such as, for example, enhanced cellular uptake, enhanced affinity for nucleic acid target and increased stability in the presence of nucleases.

The terms “subject,” “individual,” and “patient” are used interchangeably herein, and refer to an animal, for example a human, including a human in need of therapy for, or susceptible to, a condition or its sequelae. to whom treatment, including prophylactic treatment, with the pharmaceutical composition according to the present invention, is provided. The term “subject” does not exclude an individual that is normal in all respects. The term “subject” as used herein refers to human and non-human animals. The terms “non-human animals” and “non-human mammals” are used interchangeably herein and include all vertebrates, e.g., mammals, such as non-human primates (particularly higher primates), sheep, dog, rodent, (e.g., mouse or rat), guinea pig, goat, pig, cat, rabbits, cows, horses and non-mammals such as reptiles, amphibians, chickens, and turkeys.

As used herein, the terms “component,” “composition,” “composition of compounds,” “compound,” “drug,” “pharmacologically active agent,” “active agent,” “active ingredient,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

Thus, as used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Examples of such carriers or diluents include, but are not limited to, water, saline, finger's solutions, dextrose solution, and 5% human serum albumin Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In an embodiment, pharmaceutical compositions containing the therapeutic agent or agents described herein, can be, in one embodiment, administered to a subject by any method known to a person skilled in the art, such as, without limitation, orally, parenterally, transnasally, transmucosally, subcutaneously, transdermally, intramuscularly, intravenously, intraarterially, intra-dermally, intra-peritoneally, intra-ventricularly, intra-cranially, intra-vaginally, or intratumorally.

Carriers may be any of those conventionally used, as described above, and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfite), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. In one embodiment, water, preferably bacteriostatic water, is the carrier when the pharmaceutical composition is administered intravenously or intratumorally. Saline solutions and aqueous dextrose and glycerol solutions can also be employed as liquid carriers, particularly for injectable solutions.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include, without limitation, physiological saline, bacteriostatic water, Cremophor EL.™. (BASF, Parsippany, N.J.) or phosphate buffered saline (PBS). The composition should be sterile and should be fluid to the extent that easy syringeability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by using a coating such as lecithin, by maintaining the required particle size in the case of dispersion, or by using surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as appropriate, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

The compositions and formulations as described herein may be administered alone or with other biologically-active agents. Administration can be systemic or local, e.g., through portal vein delivery to the liver. In addition, it may be advantageous to administer the composition into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the therapeutic oligonucleotide locally to the area in need of treatment; this may be achieved by, for example, and not by way of limitation, local infusion during surgery, topical application, by injection, by means of a catheter, by means of a suppository, or by means of an implant.

Moreover, “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable” also includes those carriers approved by a regulatory agency of the Federal or a state government or listed in the U.S. Pharmacopeia or other generally recognized pharmacopeia for use in animals and, more particularly, in humans.

As used herein, the term “immunoglobulin G” and “IgG” refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. As used herein, the term “modified immunoglobulin G” refers to a molecule that is derived from an antibody of the “G” class. For example, the antibody is a protein consisting of one or more polypeptides substantially encoded by all or part of the recognized immunoglobulin genes. The recognized immunoglobulin genes, for example in humans, include the kappa (κ) lambda (λ) and heavy chain genetic loci, which together comprise the myriad variable region genes, and the constant region genes mu (μ) delta (δ), gamma (γ), sigma (σ) and alpha (α) which encode the IgM, IgD, IgG, IgE, and IgA isotypes or classes, respectively. The term “antibody” is meant to include full-length antibodies, and may refer to a natural antibody from any organism, an engineered antibody, or an antibody generated recombinantly for experimental, therapeutic, diagnostic or other purposes. Furthermore, full-length antibodies comprise conjugates as described and exemplified herein. Antibodies can be antagonists, agonists, neutralizing, inhibitory, or stimulatory. Specifically included within the definition of “antibody” are full-length antibodies. “Full length antibody” as used herein means the structure that constitutes the natural biological form of an antibody, including variable and constant regions.

As used herein, the term “antibody” encompasses the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains V_{L} and C_{L}, and each heavy chain comprising immunoglobulin domains V_{H}, Cγ1, Cγ2, and Cγ3. In each pair, the light and heavy chain variable regions (V_{L} and V_{H}) are together responsible for binding to an antigen, and the constant regions (C_{L}, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains V_{H}, Cγ2, and Cγ3. By “immunoglobulin (Ig)” is meant a protein having one or more polypeptides substantially encoded by immunoglobulin genes. Immunoglobulins include, but are not limited to, antibodies. Immunoglobulins may have a number of structural forms, including but not limited to full-length antibodies, antibody fragments, and individual immunoglobulin domains including but not limited to V_{H}, Cγ1, Cγ2, Cγ3, V_{L}, and C_{L}.

Depending on the amino acid sequence of the constant domain of their heavy chains, intact antibodies can be assigned to different “classes.” There are five-major classes (isotypes) of intact antibodies: IgA, IgD, IgE, IgG, and IgM, and several of these may be further divided into “subclasses”, e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known to one skilled in the art.

In one embodiment, the term “antibody” or “antigen-binding fragment” respectively refer to intact molecules as well as functional fragments thereof, such as Fab, a scFv-Fc bivalent molecule, F(ab′)₂, and Fv that are capable of specifically interacting with a desired target.

As used herein, the term “fragment antigen-binding region (Fab region)” encompasses the region of an antibody that binds to antigens. The Fab region is composed of one constant and one variable domain of each of the heavy and the light chains.

The term “Fc domain”, as used herein, encompasses the constant region of an immunoglobulin molecule. The Fc domain contains the part of an antibody that binds to the Fc receptor on immune cells. The Fc region of an antibody interacts with a number of Fc receptors and ligands, imparting an array of important functional capabilities referred to as effector functions, as described herein. For IgG, the Fc region comprises Ig domains CH2 and CH3. An important family of Fc receptors for the IgG isotype are the Fc gamma receptors (FcγRs). These receptors mediate communication between antibodies and the cellular arm of the immune system.

The “variable region” of an antibody contains the antigen binding determinants of the molecule, and thus determines the specificity of an antibody for its target antigen. The variable region is so named because it is the most distinct in sequence from other antibodies within the same isotype. The majority of sequence variability occurs in the complementarity determining regions (CDRs). There are 6 CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain specificity for a broad array of antigens.

In addition, antibodies may exist in a variety of other forms including, for example, Fv, Fab, and (Fab′)₂, as well as bi-functional (i.e., bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N. Y., 2^{nd} ed. (1984), and Hunkapiller and Hood, Nature, 323, 15-16 (1986)).

The term “epitope” refers to a region of an antigen that binds to the antibody or antigen-binding fragment. It is the region of an antigen recognized by a first antibody, where the binding of the first antibody to the region prevents binding of a second antibody or other bivalent molecule to the region. The epitope region encompasses a particular core sequence or sequences selectively recognized by a class of antibodies. In general, epitopes are comprised by local surface structures that can be formed by contiguous or noncontiguous amino acid sequences.

As used herein, the terms “selectively recognizes”, “selectively bind” or “selectively recognized” mean that binding of the antibody, antigen-binding fragment or other bivalent molecule to an epitope is at least 2-fold greater, preferably 2-5 fold greater, and most preferably more than 5-fold greater than the binding of the molecule to an unrelated epitope or than the binding of an antibody, antigen-binding fragment or other bivalent molecule to the epitope, as determined by techniques known in the art and described herein, such as, for example, ELISA or cold displacement assays.

IgGs are site-specifically modified using photoreactive antibody binding domains (AbBDs). AbBDs include Protein A, Protein G, Protein L, Protein LG, Protein LA, Protein A/G, CD4 and their subdomains, e.g., B1 domain of Protein G, engineered subdomains, e.g., Protein Z, HTB1, or sub-domains thereof.

In some embodiments, provided are AbBDs specifically binding and crosslinking to an immunoglobulin Fab region, wherein the AbBD is a variant of a Protein A domain D. In an embodiment, the AbBD is a variant of a Protein A domain D comprising an amino acid sequence with at least 80% identity to the sequence set forth in SEQ ID NO: 1 and having an amino acid replacement and/or an amino acid modification at A28, G32, Q35, S36, N46, G49, E50, K53 or L54 of SEQ ID NO: 1 or combinations thereof, wherein the AbBD is photo-reactive. In another embodiment, the AbBD is a variant of a Protein A domain D comprising an amino acid sequence with at least 90% identity to the sequence set forth in SEQ ID NO: 1 and having an amino acid replacement and/or an amino acid modification at A28, G32, Q35, S36, N46, G49, E50, K53 or L54 of SEQ ID NO: 1 or combinations thereof, wherein the AbBD is photo-reactive. In some embodiments, the AbBD is a variant of a Protein A domain D comprising an amino acid sequence set forth in SEQ ID NO: 1 and having an amino acid replacement and/or an amino acid modification at A28, G32, Q35, S36, N46, G49, E50, K53 or L54 of SEQ ID NO: 1 or combinations thereof, wherein the AbBD is photo-reactive.

In certain embodiments, provided are AbBDs specifically binding and crosslinking to an immunoglobulin IgG Fab region and/or to immunoglobulin M (IgM) Fab region, wherein the AbBD is a variant of a Protein A domain D comprising an amino acid sequence with at least 80% identity to the sequence set forth in SEQ ID NO: 1 and having an amino acid replacement and/or an amino acid modification at A28, E50, or L54 of SEQ ID NO: 1 or combinations thereof, wherein the AbBD is photo-reactive. In various embodiments, the AbBD specifically binds and crosslinks to an immunoglobulin IgG Fab region and/or to immunoglobulin M (IgM) Fab region, wherein the AbBD is a variant of a Protein A domain D comprising an amino acid sequence with at least 90% identity to the sequence set forth in SEQ ID NO: 1 and having an amino acid replacement and/or an amino acid modification at A28, E50, or L54 of SEQ ID NO: 1 or combinations thereof, wherein the AbBD is photo-reactive. In an embodiment, the AbBD specifically binds and crosslinks to an immunoglobulin IgG Fab region and/or to immunoglobulin M (IgM) Fab region, wherein is a variant of a Protein A domain D comprising an amino acid sequence set forth in SEQ ID NO: 1 and having an amino acid replacement and/or an amino acid modification at A28, E50, or L54 of SEQ ID NO: 1 or combinations thereof, wherein the AbBD is photo-reactive. In some embodiments, the pAbBD is pADE50BPA, which photo-crosslinks IgM.

In various embodiments, provided are AbBDs specifically binding and crosslinking to an immunoglobulin IgG Fab region, wherein the AbBD is a variant of a Protein L C* domain comprising an amino acid sequence with at least 80% identity to the sequence set forth in SEQ ID NO: 2 and having an amino acid replacement and/or an amino acid modification at G22 or T46 of SEQ ID NO: 2 or a combination thereof, wherein the AbBD is photo-reactive. In some embodiments, the AbBD specifically binds and crosslinks to an immunoglobulin IgG Fab region, wherein the AbBD is a variant of a Protein L C* domain comprising an amino acid sequence with at least 90% identity to the sequence set forth in SEQ ID NO: 2 and having an amino acid replacement and/or an amino acid modification at G22 or T46 of SEQ ID NO: 2 or a combination thereof, wherein the AbBD is photo-reactive. In certain embodiments, the AbBD specifically binds and crosslinks to an immunoglobulin IgG Fab region, wherein the AbBD is a variant of a Protein L C* domain comprising an amino acid sequence set forth in SEQ ID NO: 2 and having an amino acid replacement and/or an amino acid modification at G22 or T46 of SEQ ID NO: 2 or a combination thereof, wherein the AbBD is photo-reactive.

As used herein an “adapter” is conjugate molecule comprising a Fab-specific photoreactive AbBD (pAbBD) conjugated to a protein, a targeting agent of interest, an oligonucleotide, a DNA, an RNA, a detectable label, an imaging agent, a drug, a nanoparticle, a toxin, a hapten, a chelate, a polymer, a chemical handle, or a combination thereof. The Fab-specific pAbBD is conjugated to the protein, targeting agent of interest, oligonucleotide, DNA, RNA, detectable label, an imaging agent, drug, nanoparticle, toxin, hapten, chelate, polymer, a chemical handle, or a combination thereof by crosslinking the pAbBD thereto, e.g., to a Fab domain, upon exposure to UV light.

In an embodiment, the Fab-specific AbBD is a variant of the D domain of protein A or a variant of the c* domain of Protein L, wherein the Fab-specific AbBD has one or more amino acids or amino acid modifications that are adapted (i.e., by modification, such as replacement of an amino acid(s) with a photoreactive amino acid group to produce a photoreactive Fab-specific antibody binding domains (pAbBD)) that specifically binds and crosslinks to a Fab domain of an immunoglobulin upon exposure to UV light. In a particular embodiment, the immunoglobulin is IgG and the crosslinking/labelling thereof with the pAbBD is effected upon exposure to UV light. In an embodiment, the Fab-specific pAbBD having at least one photoreactive amino acid group specifically binds and crosslinks to an immunoglobulin G (IgG) Fab region upon exposure to UV light, thereby producing a Fab-specific pAbBD-labelled immunoglobulin G (IgG). In another embodiment, the Fab-specific pAbBD having at least one photoreactive amino acid group specifically binds and crosslinks to an immunoglobulin G (IgG) Fab region and/or to an immunoglobulin M (IgM) Fab region the upon exposure to UV light, thereby producing a Fab-specific pAbBD-labelled IgG and/or a Fab-specific pAbBD-labelled IgM.

The photoreactive Fab-specific antibody binding domain (pAbBD) covalently linked to/labelling the immunoglobulin G (IgG) Fab region specifically binds and crosslinks to the Fab domain of the IgG, wherein the incorporated BPAs of the pAbBD do not interfere with the photocrosslinking of the photoreactive antibody binding domains to the Fc domain. In some embodiments, the adapter photo-crosslinks to IgG. In an embodiment, the adapter photo-crosslinks to IgM, as well as to IgG. In a particular embodiment, the pAbBD is pADE50BPA, which photo-crosslinks IgM.

The Fab-specific pAbBDs may be produced with a bio-orthogonal chemical handle, e.g., biotin, azides, alkynes, thiols, at the c-terminus or fused to enzymes and/or proteins, permitting site-specific and covalent immobilization of the AbBD onto a wide range of surfaces or functionalization of IgG/IgM with other biological or chemical properties. The pAbBDs may be entirely produced recombinantly with minor variations to standard protein expression and purification methods or made synthetically (standard peptide synthesis).

In one aspect, provided herein is a conjugate molecule or an adapter comprising a protein, such as a Protein A D domain, having one or more amino acid replacement and/or an amino acid modifications that are adapted to specifically bind and crosslink to the Fab domain and the conjugate/adapter comprises the following BPA incorporation sites A28, G32, Q35, S36, N46, G49, E50, K53 or L54 of SEQ ID NO: 1 or combinations thereof. These sites allow for photo-crosslinking to IgG from various hosts.

In another aspect, provided herein is a conjugate molecule or an adapter comprising a protein, such as a Protein A D domain, having one or more amino acid replacement and/or an amino acid modifications that are adapted to specifically bind and crosslink to the Fab domain and the conjugate/adapter comprises the following BPA incorporation site at A28, E50, or L54 of SEQ ID NO: 1 or combinations thereof. This site allows for photo-crosslinking to IgG and/or IgM from various hosts. In an embodiment, the pAbBD is pADE50BPA, which photo-crosslinks IgM.

In still another aspect, provided herein is a conjugate molecule or an adapter comprising a protein, such as a Protein L C* domain, having one or more amino acid replacement and/or an amino acid modifications that are adapted to specifically bind and crosslink to the Fab domain and the conjugate/adapter comprises the following BPA incorporation sites G22 or T46 of SEQ ID NO: 2 or a combination thereof. These sites allow for photo-crosslinking to IgG from various hosts.

The following examples are presented in order to more fully illustrate certain embodiments of the invention. They should in no way be construed, however, as limiting the broad scope of the invention.

### Example 1

### IgG Conjugation by Protein A D Domain

Cetuximab (human IgG1) was photo-crosslinked with various photoreactive mutants of Protein A, D domain, under 365 nm UV illumination for 2 hrs.

### The Amino Acid Sequence of Protein A D Domain is:

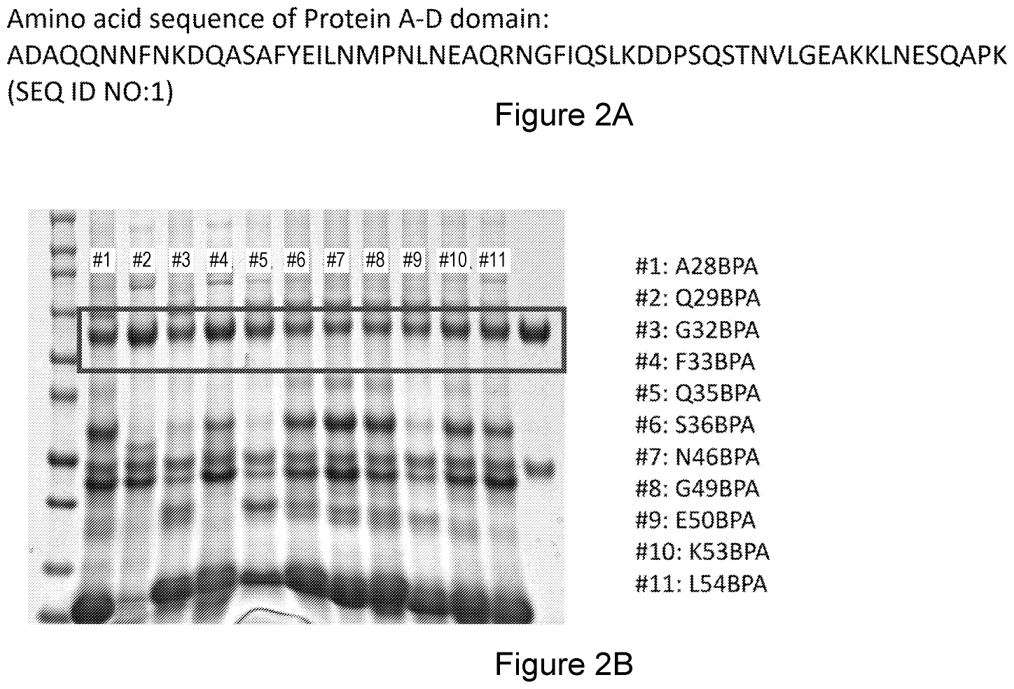

(SEQ ID NO: 1)
ADAQQNNFNKDQASAFYEILNMPNLNEAQRNGFIQSLKDDPSQSTNVLG
EAKKLNESQAPK. (FIG. 2A)

The BPA positions tested were A28, Q29, G32, F33, Q35, S36, N46, G49, E50, K53 and L54. Appreciable photo-crosslinking was observed with A28, G32, Q35, S36, N46, G49, E50, K53 and L54, as shown in the SDS-PAGE of Cetuximab (human IgG1) after photo-crosslinking with the eleven (11) listed photoreactive mutants of Protein A, D domain. (FIG. 2B)

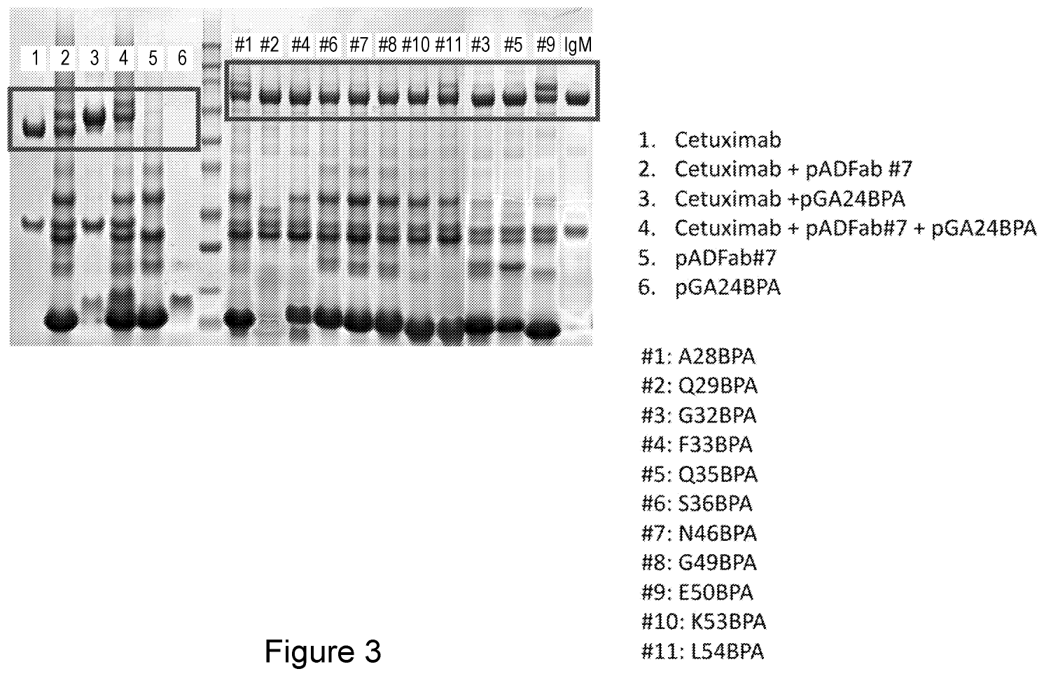

FIG. 3, left side of gel (lanes labeled 1-6) shows an SDS-PAGE of human IgG1 (Cetuximab) alone or following photo-crosslinking with pAD #7 (N46BPA), Protein G HTB1 A24BPA, or pAD #7 (N46BPA) and Protein G HTB1 A24BPA. Both domains can bind and photo-crosslink to human IgG1 simultaneously since pAD #7 (N46BPA) binds and photo-crosslinks the Fab fragment while Protein G HTB1 A24BPA photo-crosslinks the Fc domain.

FIG. 3, right side of gels (lanes labeled 1-11) shows an SDS-PAGE of IgM alone (far right lane) or after photo-crosslinking with Protein A D domain with a BPA position introduced at various sites. The BPA positions tested were A28, Q29, G32, F33, Q35, S36, N46, G49, E50, K53 and L54. Protein A D domain variants A28BPA, L54BPA, and E50BPA exhibited some photo-crosslinking to IgM.

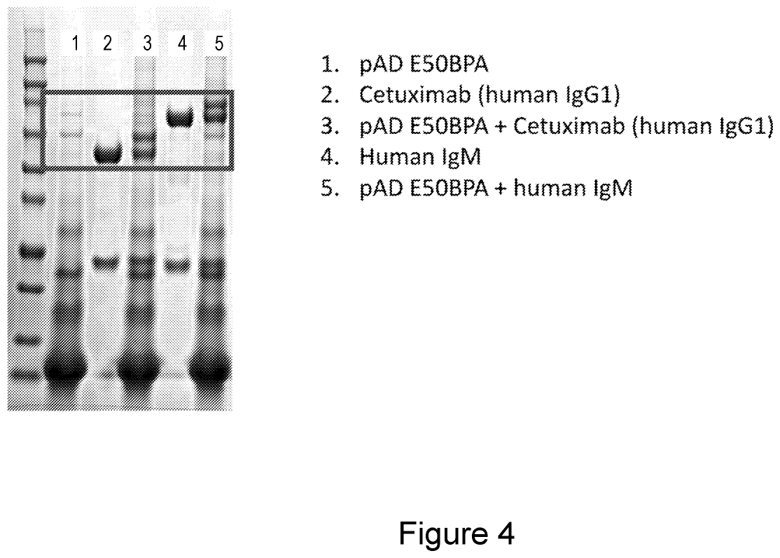

FIG. 4 shows that pAD E50BPA candidate crosslinks to Cetuximab and human IgM in the SDS-PAGE of Cetuximab (human IgG1) and human IgM alone and after photo-crosslinking with protein A D domain E50BPA. Protein A D domain E50BPA labeled ˜50% of the heavy chains of IgG and IgM.

### Example 2

### IgG Conjugation with Protein L C* Domain

### The Amino Acid Sequence of Protein L C* Domain is:

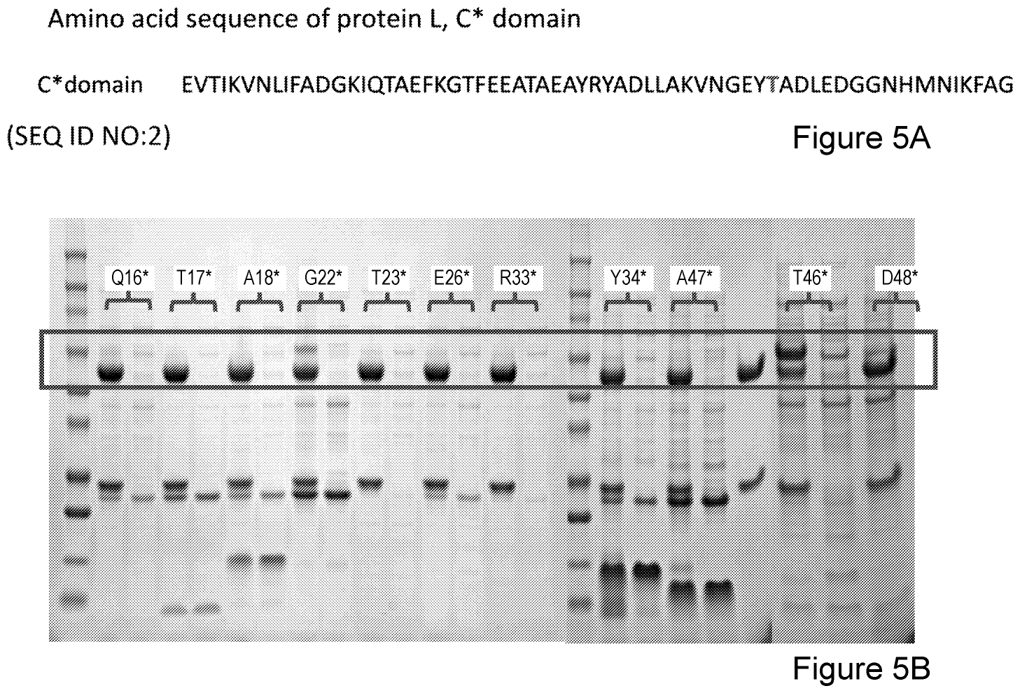

(SEQ ID NO: 2)
EVTIKVNLIFADGKIQTAEFKGTFEEATAEAYRYADLLAKVNGEYTADL
EDGGNHMNIKFAG. (FIG. 5A)

BPA was incorporated at different sites of protein L C* domain and photo-crosslinking to Cetuximab was assessed. An SDS-PAGE of Cetuximab (human IgG1) after photo-crosslinking with various photoreactive mutants of Protein L C* domain, under 365 nm UV illumination for 2 hrs. is shown in FIG. 5B. The BPA positions tested were Q16, T17, A18, G22, T23, E26, R33, Y34, A47, T46 and D48. T46BPA conjugates to human IgG1 with up to 100% conjugation efficiency. Photo-crosslinking was also observed with G22BPA.

### Example 3

### Photo-Crosslinking of IgGs from Various Host Species (Human, Rabbit, Goat) with a Photoreactive pL C* Domain with BPA Inserted at Site T46 (T46BPA)

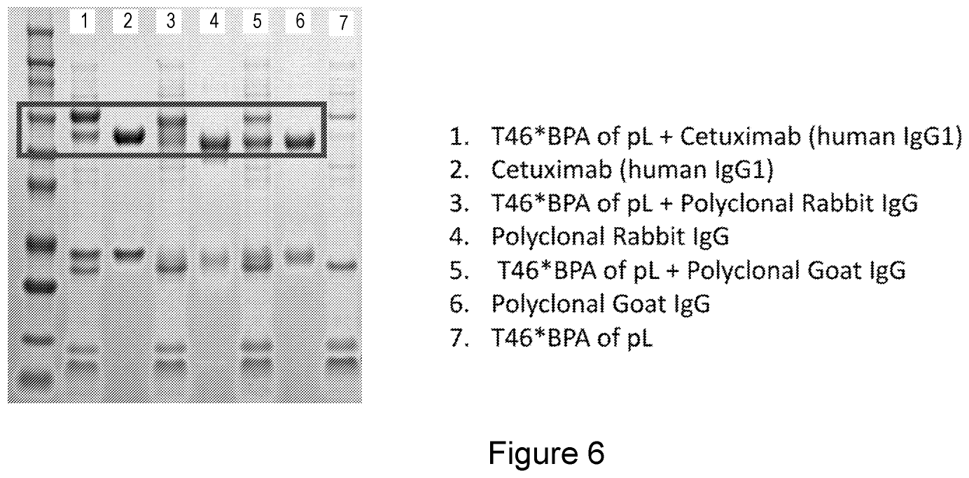

Photo-crosslinking of IgG from different host species (human, rabbit, goat) was performed under 365 nm illumination for 2 hrs. FIG. 6 shows an SDS-PAGE of the IgG from the various host species (human, rabbit, goat) alone and after photo-crosslinking with protein L C* domain T46*BPA.

The variant T46BPA of pL was shown to label human IgG1 and Rabbit IgG with close to 100% conjugation efficiency and Goat IgG with ˜50% conjugation efficiency.

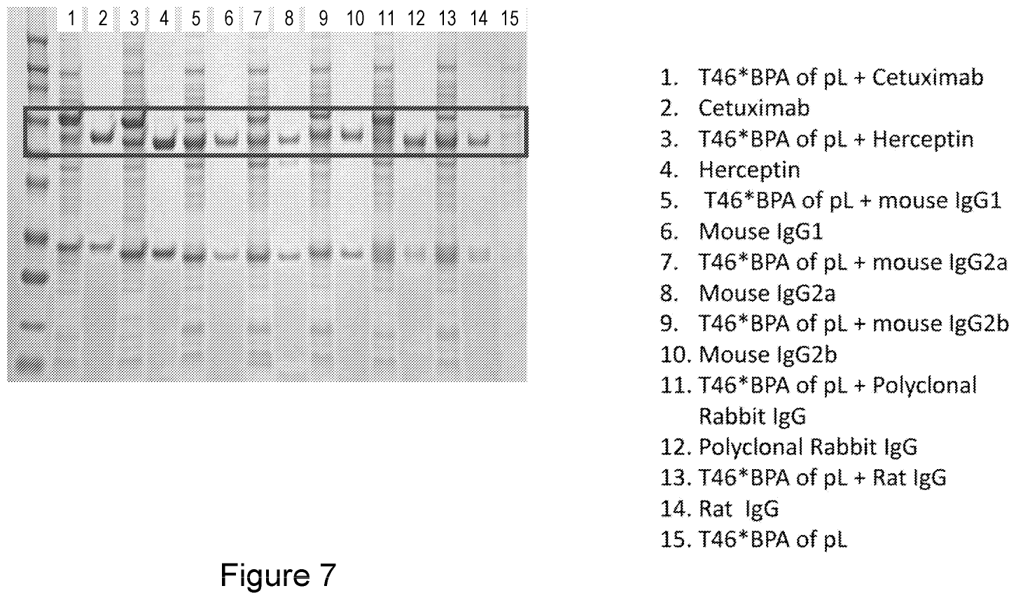

FIG. 7 shows photo-crosslinking of IgGs from various host species and subclasses with a photoreactive pL C* domain with BPA inserted at site T46 (T46BPA). The SDS-PAGE shows IgG from different host species (Cetuximab/human IgG1, Herceptin/human IgG1, mouse IgG2a, mouse IgG2b, Rabbit IgG, Goat IgG and Rat IgG) alone and after photo-crosslinking with protein L C* T46*BPA. Photo-crosslinking was performed under 365 nm illumination for 2 hrs. The variant T46BPA of pL was shown to label antibodies from a wide range of hosts and subclasses.

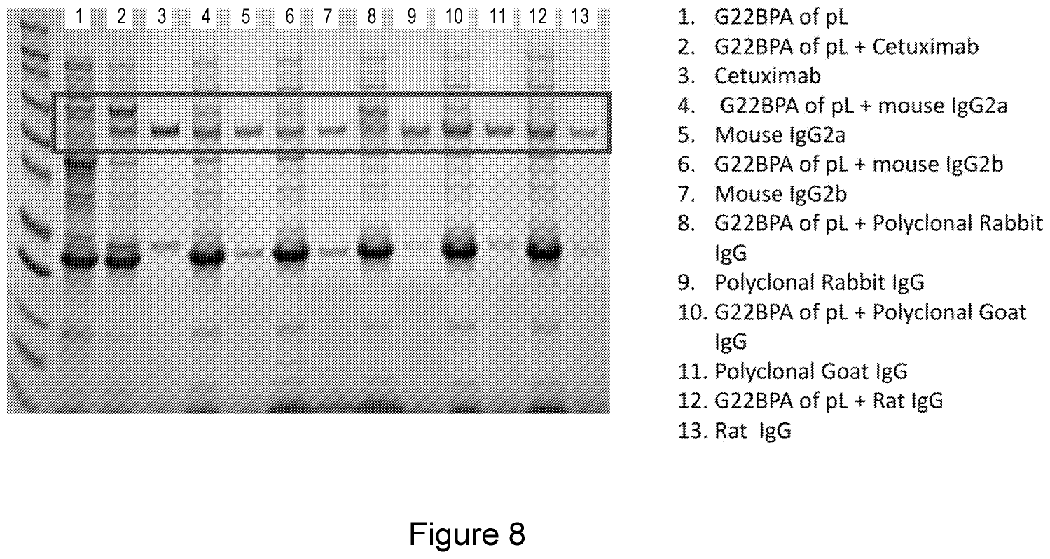

FIG. 8 shows photo-crosslinking of IgGs from various host species and subclasses (human IgG1, mouse IgG2a and 2b, rabbit, goat, and rat) with a photoreactive pL C* domain with BPA inserted at site G22 (G22BPA). The SDS-PAGE shows IgG from different host species (Cetuximab/human IgG1, mouse IgG2a, mouse IgG2b, Rabbit IgG, Goat IgG and Rat IgG) alone and after photo-crosslinking with protein L G22*BPA. Photo-crosslinking was performed under 365 nm illumination for 2 hrs. The variant G22BPA of pL was shown to label human IgG1 and Rabbit IgG with >50% conjugation efficiency.

Any patent, patent application publication, or scientific publication, cited herein, is incorporated by reference herein in its entirety.

Having described various embodiments of the invention, it is to be understood that the invention is not limited to the precise embodiments, and that various changes and modifications may be effected therein by those skilled in the art without departing from the scope or spirit of the invention as defined in the appended claims.

What is claimed is:

1. An adapter comprising an antibody binding domain (AbBD) that specifically binds and crosslinks to an immunoglobulin Fab region, wherein the AbBD is a variant of a Protein A domain D comprising an amino acid sequence with at least 80% identity to the sequence set forth in SEQ ID NO: 1 and having an amino acid replacement and/or an amino acid modification at A28, G32, Q35, S36, N46, G49, E50, K53 or L54 of SEQ ID NO: 1 or combinations thereof that is photo-reactive.

2. The adapter of claim 1, wherein the photo-reactive amino acid replacement is benzoylphenylalanine (BPA).

3. The adapter of claim 1, wherein a cysteine is engineered into the AbBD and is modified with a photo-reactive moiety.

4. The adapter of claim 1, wherein the AbBD is conjugated to a protein, a peptide, a targeting agent of interest, an oligonucleotide, a DNA, an RNA, a detectable label, an imaging agent, a drug, a nanoparticle, a toxin, a hapten, a chelate, a polymer, a chemical handle, or a combination thereof.

5. The adapter of claim 4, wherein the chemical handle is a click chemistry group selected from the group consisting of an azide, an alkyne, a constrained alkyne, dibenzocyclooctyne, a tetrazine, biotin, a thiol, and a transcyclooctene.

6. The adapter of claim 1, wherein the immunoglobulin is an IgG molecule.

7. The adapter of claim 1, wherein the amino acid replacement and/or an amino acid modification is at A28, E50, or L54 of SEQ ID NO: 1 or combinations thereof.

8. The adapter of claim 7, wherein the immunoglobulin is an IgG molecule and/or an IgM.

9. The adapter of claim 1, wherein the amino acid sequence has at least 80% identity to the sequence set forth in SEQ ID NO: 1.

10. The adapter of claim 7, wherein the photo-reactive amino acid replacement is benzoylphenylalanine (BPA).

11. The adapter of claim 7, wherein a cysteine is engineered into the AbBD and is modified with a photo-reactive moiety.

12. The adapter of claim 7, wherein the AbBD is conjugated to a protein, a peptide, a targeting agent of interest, an oligonucleotide, a DNA, an RNA, a detectable label, an imaging agent, a drug, a nanoparticle, a toxin, a hapten, a chelate, a polymer, a chemical handle, or a combination thereof.

13. The adapter of claim 12, wherein the chemical handle is a click chemistry group selected from the group consisting of an azide, an alkyne, a constrained alkyne, dibenzocyclooctyne, a tetrazine, biotin, a thiol, and a transcyclooctene.

14. The adapter of claim 9, wherein the immunoglobulin is an IgG molecule and/or an IgM.

15. An adapter comprising an antibody binding domain (AbBD) that specifically binds and crosslinks to an immunoglobulin Fab region, wherein the AbBD is a variant of a Protein L C* domain comprising an amino acid sequence with at least 80% identity to the sequence set forth in SEQ ID NO: 2 and having an amino acid replacement and/or an amino acid modification at G22 or T46 of SEQ ID NO: 2 or a combination thereof that is photo-reactive.

16. The adapter of claim 15, wherein the photo-reactive amino acid replacement is benzoylphenylalanine (BPA).

17. The adapter of claim 15, wherein a cysteine is engineered into the AbBD and is modified with a photo-reactive moiety.

18. The adapter of claim 15, wherein the Fab-specific pAbBD is conjugated to a protein, a peptide, a targeting agent of interest, an oligonucleotide, a DNA, an RNA, a detectable label, an imaging agent, a drug, a nanoparticle, a toxin, a hapten, a chelate, a polymer, a chemical handle, or a combination thereof.

19. The adapter of claim 15, wherein the chemical handle is a click chemistry group selected from the group consisting of an azide, an alkyne, a constrained alkyne, dibenzocyclooctyne, a tetrazine, biotin, a thiol, and a transcyclooctene.

20. A nucleic acid encoding the adapter according to claim 1, wherein the antibody binding domain (AbBD) comprises an amino acid sequence having at least 80% identity to the sequence set forth in SEQ ID NO: 1 and an amino acid replacement and/or an amino acid modification at A28, G32, Q35, S36, N46, G49, E50, K53 or L54 of SEQ ID NO: 1 or combinations thereof.

21. An expression vector comprising the nucleic acid of claim 20.

22. A host cell comprising the expression vector of claim 21.

23. A nanoparticle conjugate comprising the adapter of claim 1.

24. A nanoparticle conjugate comprising the adapter of claim 9.

25. A nanoparticle conjugate comprising the adapter of claim 15.

26. A nucleic acid encoding the adapter according to claim 9, wherein the antibody binding domain (AbBD) comprises an amino acid sequence having at least 80% identity to the sequence set forth in SEQ ID NO: 1 and having an amino acid replacement and/or an amino acid modification at A28, E50, or L54 of SEQ ID NO: 1 or combinations thereof.

27. An expression vector comprising the nucleic acid of claim 26.

28. A host cell comprising the expression vector of claim 27.

29. A nucleic acid encoding the adapter according to claim 15, wherein the antibody binding domain (AbBD) comprises an amino acid sequence with at least 80% identity to the sequence set forth in SEQ ID NO: 2 and having an amino acid replacement and/or an amino acid modification at G22 or T46 of SEQ ID NO: 2 or a combination thereof.

30. An expression vector comprising the nucleic acid of claim 29.

31. A host cell comprising the expression vector of claim 30.

32. A method for imaging a biological sample from a subject, the method comprising contacting the biological sample with the nanoparticle conjugate of claim 23, wherein the contacting permits imaging of the biological sample.

33. The method of claim 32, wherein the biological sample is associated with a disease.

34. The method of claim 33, wherein imaging the biological sample permits diagnosing the disease.

35. The method of claim 32, wherein the biological sample is a tissue, DNA, sperm, sera, blood, cerebrospinal fluid, a tumor, or a biomarker.

36. The method of claim 35, wherein the imaging shows abnormal tissue or the tumor, and the method further comprises treating the subject with surgery, chemotherapy, radiation therapy, bone marrow transplant, immunotherapy, hormone therapy, targeted drug therapy, cryoablation, and/or radiofrequency ablation.

37. A method for imaging a biological sample from a subject, the method comprising contacting the biological sample with the nanoparticle conjugate of claim 24, wherein the contacting permits imaging of the biological sample.

38. The method of claim 37, wherein the biological sample is associated with a disease.

39. The method of claim 38, wherein imaging the biological sample permits diagnosing the disease.

40. The method of claim 37, wherein the biological sample is a tissue, DNA, sperm, sera, blood, cerebrospinal fluid, a tumor, or a biomarker.

41. The method of claim 40, wherein the imaging shows abnormal tissue or the tumor, and the method further comprises treating the subject treating the subject with surgery, chemotherapy, radiation therapy, bone marrow transplant, immunotherapy, hormone therapy, targeted drug therapy, cryoablation, and/or radiofrequency ablation.

42. A method for imaging a biological sample from a subject, the method comprising contacting the biological sample with the nanoparticle conjugate of claim 25, wherein the contacting permits imaging of the biological sample.

43. The method of claim 42, wherein the biological sample is associated with a disease.

44. The method of claim 43, wherein imaging the biological sample permits diagnosing the disease.

45. The method of claim 42, wherein the biological sample is a tissue, DNA, sperm, sera, blood, cerebrospinal fluid, a tumor, or a biomarker.

46. The method of claim 45, wherein the imaging shows abnormal tissue or the tumor, and the method further comprises treating the subject treating the subject with surgery, chemotherapy, radiation therapy, bone marrow transplant, immunotherapy, hormone therapy, targeted drug therapy, cryoablation, and/or radiofrequency ablation.
